(19) United States
(12) Patent Application Publication
(10) Pub. No.: US 2006/0280689 A1
(43) Pub. Date: Dec. 14, 2006

(54) New MRI technique based on electron spin resonance and nitrogen endohedral C60 contrast agent

(75) Inventors: Xiao Dong Xiang, Danville, CA (US); Haitao Yang, Albany, CA (US)

Correspondence Address: QUINE INTELLECTUAL PROPERTY LAW GROUP, P.C., P O BOX 458, ALAMEDA, CA, 94501, US

(73) Assignee: Intematix Corporation

(21) Appl. No.: 11/408,621

(22) Filed: Apr. 21, 2006

Related U.S. Application Data
(60) Provisional application No. 60/673,944, filed on Apr. 22, 2005.
(60) Provisional application No. 60/673,945, filed on Apr. 22, 2005.

(51) Int. Cl. A61K 49/00

(52) U.S. Cl. 424/9.34; 423/445 B; 977/846; 977/930

(57) ABSTRACT

Methods and systems for electron spin MRI (eMRI) and novel methods for fabricating N@C₆₀ fullerenes using the discovery that certain endohedral fullerenes can be used as functional paramagnetic materials exhibiting increased relaxation times. These endohedral fullerenes provide improved labels for use in electron spin resonance (ESR) detection systems.

### CROSS REFERENCE TO RELATED APPLICATIONS

This application claims benefit of priority from provisional application 60/673,944 filed 22 Apr. 2005.

This application claims benefit of priority from provisional application 60/673,945 filed 22 Apr. 2005.

This application incorporates by reference U.S. patent application U.S. Ser. No. 11/351,312 titled ENDOHEDRAL FULLERENES AS SPIN LABELS AND MRI CONTRAST AGENTS, Atty. Docket No. 318-002110US filed Feb. 8, 2006 and provisional application U.S. Ser. No. 60/652,288, Atty. Docket No. 318-002100US filed Feb. 10, 2005, both incorporated herein by reference in their entirety for all purposes.

### FIELD OF THE INVENTION

This invention is related to the development of effective fabrication of electron spin labels that can be used in a variety of imaging applications and to methods and systems for electron spin magnetic resonance imaging (MRI).

### BACKGROUND OF THE INVENTION

The discussion of any work, publications, sales, or activity anywhere in this submission, including in any documents submitted with this application, shall not be taken as an admission that any such work constitutes prior art. The discussion of any activity, work, or publication herein is not an admission that such activity, work, or publication existed or was known in any particular jurisdiction.

Magnetic resonance imaging (MRI) has become a preferred medical imaging technique due to its non-invasive nature and high resolution. Conventional MRI is based on the detection of the nuclear spin resonance of hydrogen in human body. Although non-contrast agents (CAs) based MRI produces excellent soft-tissue images, early experiments showed that contrast agents can increase the diagnostic value immensely. Consequently, in parallel with the development of the MRI technique, there has been an explosive growth in interest in CAs, and about a third of the MRI scans are now made after administering contrast agents. The most effective CA is the Gd(III)-aquo ion, but it is not soluble at physiological pH; and more importantly, it is toxic. These problems can be overcome by sequestering the metal ion with a strong chelator. Usually the free organic ligands are toxic as well, but the highly stable lanthanide complexes have tolerable toxicity in the doses applied for MRI (0.1-0.5 mmol/kg). This explains why the majority of MRI scans still do not use CAs even though CAs provide many advantages in imaging quality and diagnostic value. Another major drawback of current MRI technology is its high cost due to the expensive liquid-helium cooled superconducting magnet required to provide a very strong magnetic field in the volume of a human body to obtain high sensitivity of NMR detection.

### SUMMARY OF THE INVENTION

This invention pertains to the use of paramagnetic endohedral fullerenes as MRI contrast agents, though in specific embodiments, paramagnetic endohedral fullerenes as prepared and described herein can also be used as spin labels, bio-reporters, and the like, as described in related applications.

The present invention overcomes several drawbacks of MRI by use of a novel contrast agent (CA), paramagnetic endohedral fullerenes (for example, N@C₆₀, representing a nitrogen atom caged inside a C₆₀ molecule) and based on the detection of the electron spin resonance (ESR) of endohedral fullerene CAs. The ESR of ordinary materials has very short relaxation time compared to NMR of hydrogen (6-8 orders of magnitudes shorter, generally less than nanoseconds), which exclude most ordinary materials from being effective contrast agents and excludes ESR from being a viable MRI technique. However, for example, paramagnetic N atoms inside highly symmetric C₆₀ cages can interact with their surroundings only through very weak electronic wave function overlaps or charge transfer, and the electron spin resonance relaxation time has been found to be very long (˜ms). As a consequence, the resonance peak is very sharp and comparable to that of NMR and the present invention makes use of endohedral fullerene contrast agents and ESR based MRI for imaging.

According to specific embodiments of the invention, endohedral fullerene contrast agents and ESR based MRI (eMRI) has a zero background positive contrast (signal change from zero to positive values), in comparison with a 100% background negative contrast (signal change from 100% to e.g. 50%) in NMR based MRI techniques. The signal to noise ratio can be high with very low concentration of CAs. The CAs dosage is simply determined by instrument sensitivity of spin detection, while the dosage of conventional NMR based MRI T₂ CAs is determined by how much CAs can lower the T₂ from a 100% value of tissues to a value that gives enough negative contrast. The fluctuation (T₂ in different tissues can vary up to 50%) in high background of conventional MRI gives rise to a high value of effective noise in the contrast image while no fluctuation exists in the zero background of eMRI according to specific embodiments of the invention. On the other hand, the noise in the image of eMRI generally comes only from Johnson noise of the electronics.

Thus, according to specific embodiments of the invention, the present invention provides one or more advantages of the new contrast mechanism and CAs: (1) increased sensitivity as discussed above, and (2) as a consequence one to three orders of magnitude decrease in required concentration of CAs; (3) it is non-toxic, since the basic ingredients of CAs are C and N; numerous studies have demonstrated that C₆₀ is not toxic to humans; (4) decreased instrumentation cost by at least one order of magnitude due to a much lower required magnetic field; (5) possibility of constructing portable instruments due to lower required magnetic field.

Spin Resonance

In NMR/MRI and ESR, spin resonance occurs when the microwave or RF frequency, and magnetic field satisfy the following equation
hv=gμB, (1)
where h is Plank's constant, v is the spin resonant frequency, B is the external magnetic field, g is the Landre Factor of the materials, and μ is the nuclear magneton: 1N for nuclear magnetic resonance (NMR/MRI), or the Bohr magneton, μ_{B}, for electron spin resonance (ESR). Nuclear spins or electron spins absorb RF/microwave energy at the spin resonance and jump between Zeeman energy levels split by an external magnetic field B.

The spin resonance signal intensity depends on two factors, the resonant frequency and the spin population difference between the split Zeeman levels, which is governed by Boltzmann statistics:

$$Δ n = 1 - exp ( - \frac{hv}{kT} ) . \qquad ( 2 )$$

At room temperature and in a 5T magnetic field, this corresponds to a factor of 10⁻⁵ reduction in resonance signal for a typical NMR. Since the electron magneton μ_{B} is about 650 times higher than the nuclear magneton μ_{N}, at the same temperature and magnetic field, the electron spin resonance energy (frequency) is much higher than that of NMR, and therefore the spin population difference of ESR is also much higher. This gives a much stronger (˜10⁶ times) ESR signal than NMR at the same temperature and magnetic field, and has a profound effect on the sensitivity of detection.

Unfortunately, the major problem in ESR imaging is the short relaxation time (usually on the order of ns, 6-8 orders of magnitude shorter than that of NMR) or broad line width of electron spin resonance. This is because, usually, the electron wave functions in solids are sufficiently overlapped to cause the spins of individual electrons to be disturbed or quenched by the electrostatic fields of the surrounding environment. With random noise (Johnson noise) being distributed over a broad frequency range, signal to noise ratio or sensitivity can be dramatically degraded in broad peak detection. Furthermore, short relaxation times increase the difficulty or even prevent the adoption of time resolved pulse techniques widely and successfully used in NMR spectroscopy. These two are the reasons why ESR techniques have heretofore been considered less useful for biomedical research and diagnostics.

Software Component Implementations

Various embodiments of the present invention provide methods and/or systems for eMRI imaging and control functions that can be implemented on a general purpose or special purpose information handling appliance using a suitable programming language such as Java, C++, Cobol, C, Pascal, Fortran., PL1, LISP, assembly, etc., and any suitable data or formatting specifications, such as HTML, XML, dHTML, TIFF, JPEG, tab-delimited text, binary, etc. In the interest of clarity, not all features of an actual implementation are described in this specification. It will be understood that in the development of any such actual implementation (as in any software development project), numerous implementation-specific decisions must be made to achieve the developers' specific goals and subgoals, such as compliance with system-related and/or business-related constraints, which will vary from one implementation to another. Moreover, it will be appreciated that such a development effort might be complex and time-consuming, but would nevertheless be a routine undertaking of software engineering for those of ordinary skill having the benefit of this disclosure.

Other Features & Benefits

The invention and various specific aspects and embodiments will be better understood with reference to the following drawings and detailed descriptions. For purposes of clarity, this discussion refers to devices, methods, and concepts in terms of specific examples. However, the invention and aspects thereof may have applications to a variety of types of devices and systems. It is therefore intended that the invention not be limited except as provided in the attached claims and equivalents.

Furthermore, it is well known in the art that complex imaging systems and methods and chemical fabrication methods such as described herein can include a variety of different components and different functions in a modular fashion. Different embodiments of the invention can include different mixtures of elements and functions and may group various functions as parts of various elements. For purposes of clarity, the invention is described in terms of systems that include many different innovative components and innovative combinations of innovative components and known components. No inference should be taken to limit the invention to combinations containing all of the innovative components listed in any illustrative embodiment in this specification.

All references, publications, patents, and patent applications cited herein are hereby incorporated by reference in their entirety for all purposes.

### DEFINITIONS

The term “fullerene” is used generally herein to refer to any closed cage carbon compound containing both six- and five-member carbon rings independent of size and is intended to include the abundant lower molecular weight C₆₀ and C₇₀ fullerenes, larger known fullerenes including C₇₆, C₇₈, C₈₄, C₉₂, C₁₀₆ and higher molecular weight fullerenes C_{2N} where N is 50 or more (giant fullerenes) that can be nested and/or multi-concentric fullerenes. The term is intended to include “solvent extractable fullerenes” as that term is understood in the art (generally including the lower molecular weight fullerenes that are soluble in toluene or xylene) and to include higher molecular weight fullerenes that cannot be extracted, including giant fullerenes that can be at least as large as C₄₀₀. The term fullerenes additionally include heterofullerenes in which one or more carbons of the fullerene cage are substituted with a non-carbon element (e.g., B, N, etc.) and derivatized/functionalized fullerenes.

Endohedral fullerenes are fullerene cages that encapsulate an atom or atoms in their interior space. They are written with the general formula Mₘ@C₂ₙ, where M is an element, m is the integer 1, 2, 3, 4, 5, or higher, and n is an integer number. The “@” symbol refers to the endohedral or interior nature of the M atom inside of the fullerene cage. Endohedral fullerenes corresponding to most of the empty fullerene cages have been produced and detected under varied conditions. Endohedral metallofullerenes include, but are not limited to those where the element M is a lanthanide metal, a transition metal, an alkali metal, an alkaline earth metal, and a radioactive metal.

The terms “derivatization” or “functionalization” generally refer to the chemical modification of a fullerene or the further chemical modification of an already derivatized fullerene. Such chemical modification can involve the attachment, typically via covalent bonds, of one or more chemical groups to the fullerene surface. Further derivatization of a derivatized fullerene refers to further attachment of groups to the fullerene surface.

A “a paramagnetic material caged within a fullerene” refers to a material that when present within an endofullerenes is paramagnetic. The material can be paramagnetic when not caged within the fullerene (e.g, a paramagnetic material) or it can include a material that is not paramagnetic when outside the fullerene, but when caged within the fullerene, the endofullerene is paramagnetic.

The term “nanoparticle”, as used herein refers to a particle having at least one dimension equal to or smaller than about 500 nm, preferably equal to or smaller than about 100 nm, more preferably equal to or smaller than about 50 or 20 nm, or having a crystallite size of about 10 nm or less, as measured from electron microscope images and/or diffraction peak half widths of standard 2-theta x-ray diffraction scans.

### BRIEF DESCRIPTION OF THE DRAWINGS

FIG. 1 is a diagram showing of an example nitrogen endohedral fullerene according to specific embodiments of the invention.

FIG. 2 is a diagram showing an example production method of a nitrogen endohedral fullerene (e.g., N®C₆₀) according to specific embodiments of the invention.

FIG. 3 is a diagram of an example instrument setup for spin resonance detection according to specific embodiments of the invention.

FIG. 4 is an illustration of a pulse sequence generated by an ultra fast pulse sequence generator according to specific embodiments of the invention.

FIG. 5 is a block diagram of am eMRI system according to specific embodiments of the invention.

FIG. 6 illustrates an example of an instrument set-up that can be used for human body eMRI according to specific embodiments of the invention.

FIG. 7 illustrates typical gradient coils used to generate field gradient along x, y, z directions according to specific embodiments of the invention.

FIG. 8 illustrates a typical two-shot interleaved epi sequence: a) pulse sequence diagram; b) k-space coverage diagram according to specific embodiments of the invention.

FIG. 9 is a block diagram showing a representative example logic device in which various aspects of the present invention may be embodied.

### DESCRIPTION OF SPECIFIC EMBODIMENTS

Before describing the present invention in detail, it is to be understood that this invention is not limited to particular compositions or systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content and context clearly dictates otherwise. Thus, for example, reference to “a device” includes a combination of two or more such devices, and the like.

### 1. NEW MRI TECHNIQUE BASED ON ELECTRON SPIN RESONANCE AND NITROGEN ENDOHEDRAL C₆₀ CONTRAST AGENT

The present invention according to specific embodiments uses nitrogen endohedral fullerenes as the ESR imaging contrast agents. Unlike electron spin resonance of regular materials, the generally spherical fullerene skeleton (such as C₆₀) forms a Faraday cage for the enclosed paramagnet (in particular a nitrogen atom) and there is essentially no charge transfer to the surrounding fullerene, for example as shown in FIG. 1. The nitrogen atom is located in the center of the fullerene and its electron wave function is confined within the fullerene, effectively isolated from the environmental perturbation, resulting in a significantly enhanced relaxation time. ESR measurements of N@C₆₀ have shown extremely narrow spin resonance line width, corresponding to a relaxation time on the order of ms which is comparable to that of hydrogen NMR signals with CAs.

With N@C₆₀ as ESR contrast agents, the disadvantage of short relaxation time in ESR can be overcome, and higher sensitivity and much lower magnetic field (and hence lower cost) over MRI techniques can be achieved. Furthermore, in an N@C₆₀ ESR contrast agent imaging, the ESR signal only comes from the contrast agents. There is basically no background signal from other regions without contrast agents. The image contrast ratio can be very high even with a small ESR signal. This is in sharp contrast to MRI imaging: contrast agents are used in MRI to enhance the NMR relaxation and therefore lower the NMR signal. The image is acquired based on a high (100%) background, and the contrast ratio depends on how much the NMR signal can be lowered.

Further, it is reported that a nitrogen atom implanted fullerene produces a paramagnetic center with hyperfine interaction properties very close to that of atomic nitrogen (Almeida Murphy et al. (1996) Phys. Rev. Lett. 77: 1075). The paramagnetic complex is soluble in organic solvents, is stable at room temperature, and withstands exposure to air. The almost spherical fullerene skeleton (like C₆₀ or C₇₀) forms a Faraday cage for the atom that is implanted inside. The paramagnetic atom sits almost exactly at the center, without charge transfer to the cage, the structure of the cage is not distorted and the electronic wave function of the paramagnetic atom is confined within and therefore isolated from the environment. Thus, the relaxation time of this paramagnetic complex is very long (a few hundreds microseconds), which is close to that of NMR specimens.

In certain embodiments, the fullerene is a C₆₀ fullerene. Fullerene C₆₀ is a spherically π-conjugated all carbon molecule that can accept six electrons successively in solution (Hirsch (1994) The Chemistry of the Fullerenes, Thieme, New York; Wie et al. (1992) J. Am. Chem. Soc. 114: 3978). The C₆₀ can be directly attached to carbon, nitrogen, and iridium elements, and the like. Thus the endohedral fullerenes can be directly attached to organic or inorganic molecules at specific position for use as electron spin labels.

### 2. eMRI OF THE INVENTION

Because the ESR relaxation times (T₁ and T₂) of the contrast agent of the invention are comparable to the values of NMR materials (e.g. protons in water), a variety of magnetic resonance imaging (MRI) technique based on NMR can be directly used in prototype and operational ESR imaging systems (eMRI).

Compared with the conventional nuclear MRI (nMRI), eMRI has several important advantages, which are summarized below:
a) Higher sensitivity due to the ability to use a higher resonance frequency;
b) Lower cost due to a much lower required magnetic field.
c) Higher spatial resolution due to lower magnetic field if field gradient is kept constant;
d) Shorter performance time due to the same reason.

There are two major concerns in nMRI: one is the sensitivity, the other is the performance time. Due to the low inherent sensitivity of NMR, it takes longer scan time to increase the sensitivity of MRI imaging, contrast agents are used to enhance the NMR relaxation and therefore adversely lower the NMR signal. The image is acquired based on a high (100%) background, and the contrast ratio depends on how much the NMR signal can be lowered. In medical applications, however, the available time is often limited by the object under investigation.

There are two major concerns in nMRI: one is the sensitivity, the other is the performance time. Due to the low inherent sensitivity of NMR, it takes longer scan time to increase the sensitivity of MRI imaging, contrast agents are used to enhance the NMR relaxation and therefore adversely lower the NMR signal. The image is acquired based on a high (100%) background, and the contrast ratio depends on how much the NMR signal can be lowered. In medical applications, however, the available time is often limited by the object under investigation.

To decrease the performance time of nMRI without sacrificing sensitivity, each hardware component of a nMRI system has to work at maximum capacity and has little room for further improvement. One major limitation of fast imaging is the gradient coils and their driving electronics. Modern MR Imagers all use spatial encoding techniques to realize 2D or 3D imaging, which apply a series of magnetic field gradient pulse sequences to the object. The performance time is approximately the sum of the duration times (t) of all these gradient pulses. The spatial resolution (or pixel size, resolution (or pixel size, ΔX) of the image, on the other hand, is determined by the product (more accurately the integral product) of the magnetic field gradient amplitudes (G) and the gradient pulse duration (t), which can be expressed as [10]:

$$Δ X = \frac{1}{γ \cdot t \cdot G} = \frac{{B}_{0}}{ω \cdot t \cdot G} \qquad ( 3 )$$

where γ is the gyromagnetic ratio, ω is the MR frequency, and B₀ is the required DC magnetic field. The equation indicates that spatial resolution is proportional to the DC magnetic field B₀ and inversely proportional to the performance time and the field gradient. To decrease the performance time which high spatial resolution, a very high gradient field (G) has to be generated with very high slew rate in order to generate the required pulse with short duration (t). It is a common knowledge that the driving voltage/current amplitude and the slew rate are in conflict with each other and it is very hard to improve if the hardware limitation has been reached.

According to specific embodiments of the invention, using similar techniques, the performance time and the spatial resolution of eMRI is governed by the same Eq. (3). However, there is a very important difference between eMRI and nMRI, the gyromagnetic ratio (γₑ) of electron spin is 650 times higher than that of the proton (γ_{N}) in nMRI. Larger γₑ relative to γ_{N} creates many advantages. First, since the achievable magnetic field is no longer the limitation of eMRI frequency as opposed to that in nMRI, one order of magnitude increase in imaging frequency (limited here by transparency of human body to the imaging frequency ˜1 GHz) is achieved. This in turn increases the sensitivity of eMRI by two orders of magnitude. After selecting the higher frequency ˜1 GHz, there is still room to lower the magnetic field by about two orders of magnitude to 350 Gauss compared to that of nMRI (most common ˜3.9T).

According to specific embodiments of the invention, a lowered B₀ field in eMRI provides one of following advantages:
a) The performance time of eMRI can be decreased by about two orders of magnitude when the spatial resolution and the gradient field amplitude are kept the same as nMRI.
b) The spatial resolution of eMRI can be improved by two orders of magnitude when the performance time and the gradient field amplitude are kept the same as nMRI.
c) The gradient field amplitude required for eMRI can be decreased by two orders of magnitude when the spatial resolution and the performance time are kept the same as nMRI, which significantly lowers the cost.
d) In a possibly more likely configuration, the spatial resolution will be kept the same as nMRI. The performance time will be decreased by one order of magnitude to enhance the capability of eMRI over nMRI; and the gradient field amplitude will be decreased by one order of magnitude to lower the cost of the instrument.

### 3. AN ESTIMATION OF CAs DOSAGE FOR eMRI

According to specific embodiments of the invention, the detection electronics used for nMRI can be adopted to do ESR imaging with minor modifications. Regular MRI works in the RF frequency around 100 to 200 MHz, limited by the availability of the magnetic field generated by a superconducting magnet large enough in size for a human body. With ESR imaging according to the invention, the spin resonance frequency can be raised up to 1 GHz (this frequency has been used for highest field non-imaging NMR) with only a several hundred Gauss magnetic field. Due to the increase of the resonance frequency and therefore the increase of spin population difference, the sensitivity of ESR contrast agents will increase by at least one order of magnitude, which gives ˜10¹¹ to 10¹³ electron spins sensitivity, as compared with spin sensitivity of about 10¹² to 10¹⁴ proton spins in conventional MRI detection.

To reach the typical whole body MRI resolution of around 1 mm, the concentration of ESR contrast agents according to specific embodiments of the invention is about:

$$C = \frac{{10}^{11} \sim {10}^{13}}{{( 1 mm )}^{3}} = 1.6 \times {10}^{- 7} \sim 1.6 \times {10}^{- 5} mol / L \qquad ( 4 )$$

which is one to three orders of magnitude lower than regular MRI contrast agent concentration (0.1 to 0.5 mmol/L). Note that NMR based MRI CAs dosage is not determined by spin detection sensitivity but rather a dosage that can quench all surrounding hydrogen T₂ to yield enough negative contrast. Given a same signal to noise ratio, eMRI of the invention requires a much lower concentration than conventional CA concentration.

### 4. MAKING A PARAMAGNETIC FULLERENE (e.g., C₆₀) SOLUBLE IN WATER

For N@C₆₀ or other fullerenes to be used as CAs in most physiological applications, it is desirable to be dissolved in water first. As is well known, pure C₆₀ can be easily dissolved in hydrocarbon solvents such as toluene, however since C₆₀ is hydrophobic it cannot be dissolved in water without surface modification. In specific embodiments, the invention uses a method to prepare water soluble fullerenes (e.g., C₆₀) by embedding them in large spherical water soluble host molecule.

One example method according to specific embodiments of the invention involves embedding N@C₆₀ inside a γ-cyclodextrin molecule as has been demonstrated for C₆₀. γ-cyclodextrin is not toxic due to its origin from corn and it is soluble in water. Using γ-cyclodextrin to enclose N@C₆₀ inside its structure enables N@C₆₀ to be dissolved into water as a complex with γ-cyclodextrin. At concentrations of 0.02 mol/L for γ-cyclodextrin and ˜8×10⁻⁵ mol/L for N@C₆₀, a complex of monomeric N@C₆₀ with each γ-cyclodextrin magenta solution can be obtained via reflux. At a low ratio of γ-cyclodextrin to N@C₆₀, a cluster of several N@C₆₀ molecules surrounded by γ-cyclodextrin is formed (yellow solution).

### 5. MANUFACTURE OF NITROGEN ENDOHEDRAL FULLERENE CONTRAST AGENTS

Traditionally endohedral fullerenes are prepared by adding the appropriate materials during the formation of the fullerenes. However, since N₂ is reactive, only ion implantation during C₆₀ sublimation has been successfully used for producing N@C₆₀ and P@C₆₀. However, since the exposure time of C₆₀ vapor to the N is very short, and the molecular concentration of C₆₀ in the vapor phase is very low, and after C₆₀ is deposited it is covered by other C₆₀ solid molecules, the chances of N or P ions entering into the C₆₀ cage is very low. As a consequence, the current fabrication technique suffers from extremely low yields [Wendt M, Wacker F, Wolf K J, et al, “[Keyhole-true FISP: fast T2-weighted imaging for interventional MRT at 0.2 T]”, Rofo Fortschr Geb Rontgenstr Neuen Bildgeb Verfahr 170, 391 (1999), German; T. Almeida Murphy, Th. Pawlik, A. Weidinger, M. Höhne, R. Alcala, and J.-M. Spaeth, Phys. Rev. Lett. 77, 1075 (1996).]. The highest reported endohedral ratio so far is in the range of 10⁻⁴ to 10⁻⁵. This kind of low yield makes it extremely difficult to consider large scale applications of endohedral fullerenes.

The present invention in specific embodiments involves a new technique that allows highly efficient fabrication of endohedral fullerenes with much higher concentration level than previous reported techniques. This method involves inductively induced Nitrogen ion plasma inside a sealed chamber (e.g., a tube) of glass, quartz, or other suitable material filled with high concentration of C60 or other fullerene molecule vapor.

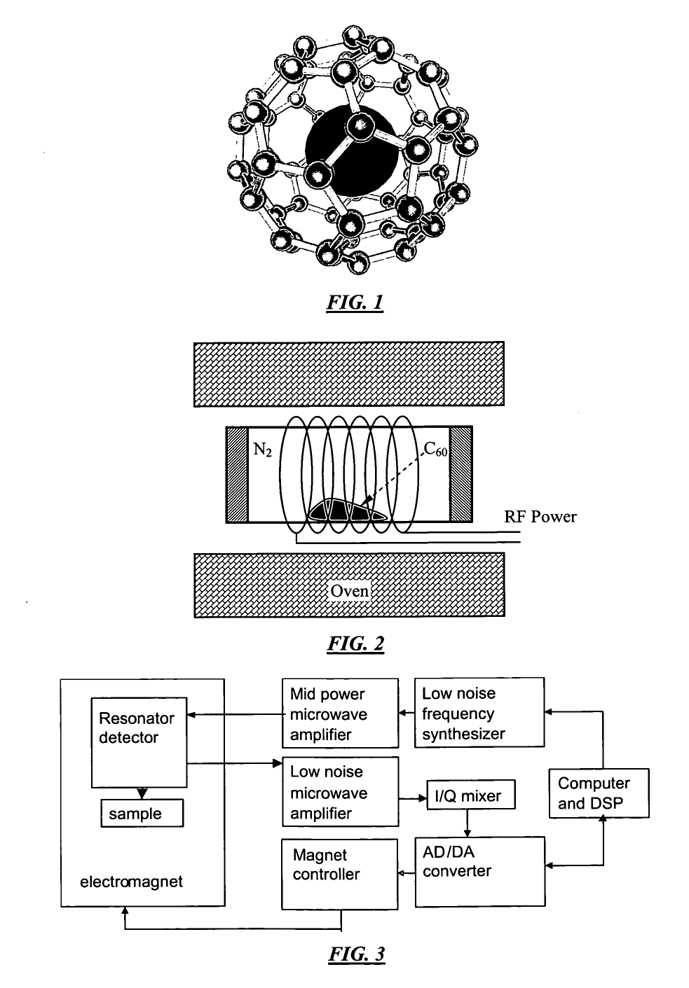

FIG. 2 is a diagram showing an example production method of a nitrogen endohedral fullerene (e.g., N@C60) according to specific embodiments of the invention. C₆₀ powder and N₂ gas are sealed within a quartz (or glass or other suitable material) tube, which is surrounded with a RF coil. To seal a large amount of N₂ gas inside a quartz or other material tube, one end of the tube is cooled by liquid N₂ to condense enough N₂ gas inside the tube while keeping the pressure inside the tube lower than atmosphere. This helps the sealing of the tube with a high temperature torch. The whole system is then put into an oven or otherwise heated to about 450° C. Solid C₆₀ will vaporize under 450° C. filling the entire tube, and inductively induced nitrogen ions will collide with C₆₀ molecules continuously in the process.

In specific embodiments, the longer the system is operated, the higher concentration of N@C₆₀ is obtained. Inductively induced ion plasma instead of high electric field induced plasma (as in previous studies) reduces the chance of fracturing C₆₀ in the process. After the process, the quartz tube is cooled. The nitrogen endohedral fullerene powder (mixed with empty C₆₀) will be extracted from the quartz tube by an appropriate chemical solution (such as toluene or hexane).

Endohedral fullerenes as used in an eMRI according to specific embodiments of the invention can be produced by any of a number of other methods known to those of skill in the art, though a presently preferred method is as described above. Other approaches are discussed in above-referenced patent applications.

### 6. SEPARATION OF N@C₆₀ FROM PURE C₆₀

High pressure liquid chromatography (HPLC) techniques have been successfully used to separate N@C₆₀ from pure C₆₀ [B. Pietzak, M. Waiblinger, T. Almeida Murphy, A. Weidinger, M. Höhne, E. Dietel, A. Hirsch, Chem. Phys. Lett. 279, 259 (1997).]. However, this technique is very slow and not efficient for industrial production.

The present invention in specific embodiments is further involved with using simulated moving bed (SMB) chromatography to purify the endohedral fullerenes from the empty fullerenes. SMB chromatography is a continuous solid-liquid separation process that purifies two components of a feed stock [A. Grupp, O. Haufe, M. Jansen, M. Mehring, M. Panthofer, J. Rahmer, A. Reich, M. Rieger, X.-W. Wei, Structure and Electronic Properties of Molecular Nanostructures, AIP, 31 (2002).]. This process is attractive for its efficient use of separations packing and eluant and high productivity. After purification, the empty fullerenes can be reused to produce endohedral fullerenes.

Thus, according to specific embodiments of the invention, combining the above described continuous gas phase N ion plasma implantation and SMB chromatography, the invention drastically increases the yield and production quantity for N@C₆₀. It will be understood to those of skill in the art that this method can be employed for other endohedral fullerenes.

Other methods for separating the endohedral fullerenes (fullerenes containing the desired moiety) from empty fullerenes include HPLC, and other methods that are also known to those of skill in the art. In this regard it is noted that U.S. Patent Publication 2003/0157016 describes a purification method based on selective formation of cationic fullerene species by chemical protonation or addition of other cationic electrophilic groups, which is distinct from fullerene cation formation via the chemical or electrochemical oxidation. Cation formation can equally be conducted by oxidative electrochemistry or by chemical addition of a cationic agent, such as protonation by a Bronsted acid or addition of an electrophile. Photochemical cation generation methods can also be used.

### 7. CHARACTERIZE ESR PROPERTIES OF N@C₆₀ in WATER SOLUTION

In specific embodiments the invention is involved with a proprietary Microwave Electron Spin Resonance Detection system with an electromagnet field up to 1T. FIG. 3 is a diagram of an example instrument setup for spin resonance detection according to specific embodiments of the invention. The microwave frequency synthesizer and medium power amplifier provide an ultra-low-noise microwave excitation signal to excite the spins in the sample. The low-noise amplifier then picks the weak signal induced only by spin resonance, which is isolated from the strong background excitation signal, and amplifies it without adding significant noise. The amplified signal is processed by an I/Q mixer, read by A/D converters, and then analyzed with a computerized digital signal processing (DSP) system.

N@C₆₀ solution is sealed in small capillaries and measured using the above detection system. The spin resonance line width and relaxation time can thus be characterized and the concentration of the N@C₆₀ solution is optimized to get the best line width and relaxation time to meet the requirements of eMRI imaging.

### 8. ULTRA FAST PULSE SEQUENCE GENERATOR FOR ESR STUDY

As mentioned above, one advantage of the eMRI based on new contrast agent is the shorter performance time, which requires high-speed control electronics to provide the pulse sequence with shorter pulse width and time interval. The electronics of conventional nMRI only need millisecond pulses, while the eMRI benefits from microsecond pulses. Although there are some pulse generators available on the market generating pulse shorter than 1 ns, they cannot provide an adequate pulse sequence so that each pulse and the interval between pulses can be precisely controlled.

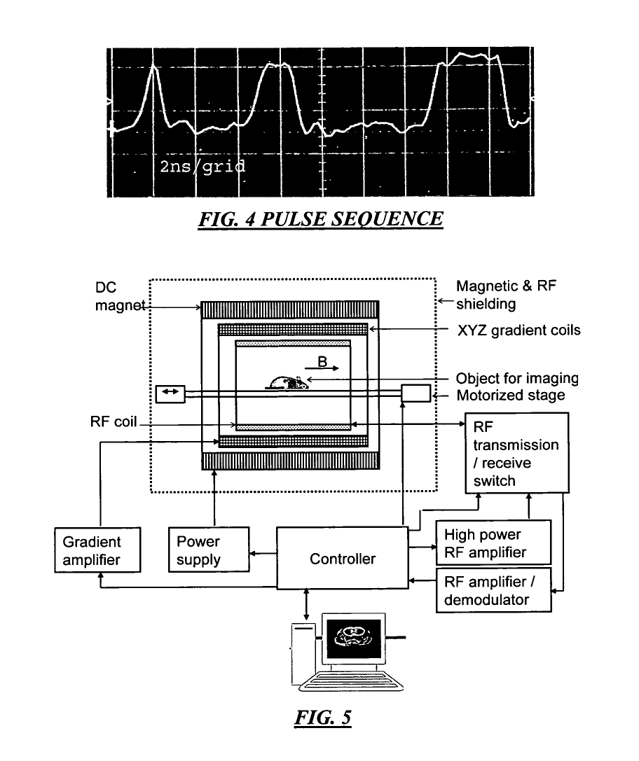

Thus, in further embodiments, systems of the invention can employ a nano-second pulse sequence generator that is specially designed for the electron spin echo observation. Both the pulse width and time interval between pulses can be adjusted from 1 ns with 10 ps resolution. FIG. 4 is an illustration of a pulse sequence generated by an ultra fast pulse sequence generator according to specific embodiments of the invention. This sequence is measured by 1.5 GHz oscilloscope (LeCroy 9362). The three pulses have Ins, 2 ns and 4 ns width with the pulse interval of 4 ns and 6 ns, respectively. The channels of controller are extendable and can be synchronized. With minor modification, the controller can be directly used for an eMRI setup as described herein.

### 9. DESIGN INSTRUMENT AND METHODOLOGY FOR 3D ESR IMAGING (eMRI)

In further embodiments, various aspects of the present invention can be combined into an eMRI machine, either in prototype or operational form.

Many kinds of MRI techniques have been developed and investigated since the 1970's. Such techniques include sensitive point technique, field focusing NMR, sensitive line method, line scan technique, echo line imaging, projection-reconstruction technique, Fourier imaging, spin-warp imaging, rotating-frame imaging, planar and multi-planar imaging and echo planar imaging (EPI) [R. R. Ernst, G. Bodenhausen, A. Wokaun, Principles of Nuclear Magnetic Resonance in One and Two Dimensions, p541, Clarendon press, Oxford, (1987).] These techniques distinguish from each other by their different encoding methods used to realize 2D or 3D imaging. In modern imagers, the slice selection, phase encoding and frequency encoding are the most popular methods to realize fast imaging with high sensitivity. The most popular MRI techniques, such as EPI, spin-wrap imaging, Fourier imaging, use one or all of these encoding method. EPI is currently the fastest imaging which can acquire one 2D image (or single slice image in 3D imaging) with 128×128 pixels within 40 ms.

In various embodiments of the present invention, an eMRI system based on aforementioned encoding methods is constructed. In the example discussed herein, the focus is on demonstrating some of the advantages discussed above: higher image acquisition rate and higher sensitivity (or lower contrast agent dosage) with the same spatial resolution as nMRI. The introduction of the above mentioned encoding method will be described in gradient coil and amplifier section.

Similar to any nMRI system, the basic hardware components of the eMRI are the magnet for producing a stable magnetic field, the gradient coils for creating a variable field, radio frequency (RF) coils used to transmit energy and to encode spatial position, and electronics that drive the magnet and coils, as well as computer controlled scanning operation and data processing. Since the eMRI needs magnetic field of several hundred gauss, magnetic shielding is required to avoid the disturbance from surrounding environment. FIG. 5 is a block diagram of am eMRI system according to specific embodiments of the invention. The detailed description of each component is discussed below.

As a further example, the present invention may be embodied in a full body human eMRI system. FIG. 6 illustrates an example of an instrument set-up that can be used for human body eMRI according to specific embodiments of the invention. The setup is similar to the conventional MRI setup. Driven by the control electronics through X, Y, Z amplifier, the gradient coil can provide a gradient magnetic field variable in three dimensions (X, Y, and Z) which permits localization of signal detection to the specific desired region of tested sample or organism (e.g., human body). The RF coil or alternatively the microwave antenna array is used as a spin resonance detection element. The gradient field can be applied so that only the section contains the interesting region is imaged. The heating element is optional and is described in above referenced patent applications.

1) Magnet System

The DC magnetic field generated by the magnet system determines the frequency of the magnetic resonance. When the frequency is 1 GHz, the required magnetic field is 350 Gauss. This field strength is easy to achieve using either a permanent magnet or an electromagnet. Permanent magnets have been successfully used in conventional MRI system as a significant approach to reduce the system cost and a permanent magnet is easier to be used in eMRI due to the low field design.

An electromagnet is another solution which has the advantage of the adjustable magnetic field. Two kinds of electromagnet can be used in the eMRI system, the iron core electromagnet and the air core solenoid electromagnet.

In a demonstration system, an electromagnet that can generate 0.4T magnetic field with 100 A driving current is used. The air gap between the iron cores are 200 mm, the iron core diameter is also 200 mm, which is proper to used in eMRI for small animal detection.

For high quality ESR imaging, the DC magnetic field has to be very uniform in the entire detection region to ensure the high signal noise ratio and avoid the image distortion. A custom-designed air core solenoid electromagnet will generate more uniform magnetic field inside the solenoid. To generate 350 Gauss, the coil current density is 30 kA/m. This requirement can be easily achieved by adding water cooling to the system. The 30V/100 A power supply with current stability of better than 100 ppm can be used to drive the magnet, which is commercially available.

2) Gradient Coils and Amplifiers

In an eMRI system according to specific embodiments of the invention, gradient coils are used to produce a linear variation in field along 3 directions respectively. A gradient field is used generally in any kind of MRI technique for the purpose of localization of the image slices as well as phase encoding and frequency encoding. The detail design of the gradient coils as used in various conventional nNMRI are found in many references [www(.)mritutor(.)org/mritutor/coils(.)htm] and are commercially available [www(.)insightneuroimaging(.)com]. In an example embodiment, these standard gradient coils are used in an eMRI system of the invention.

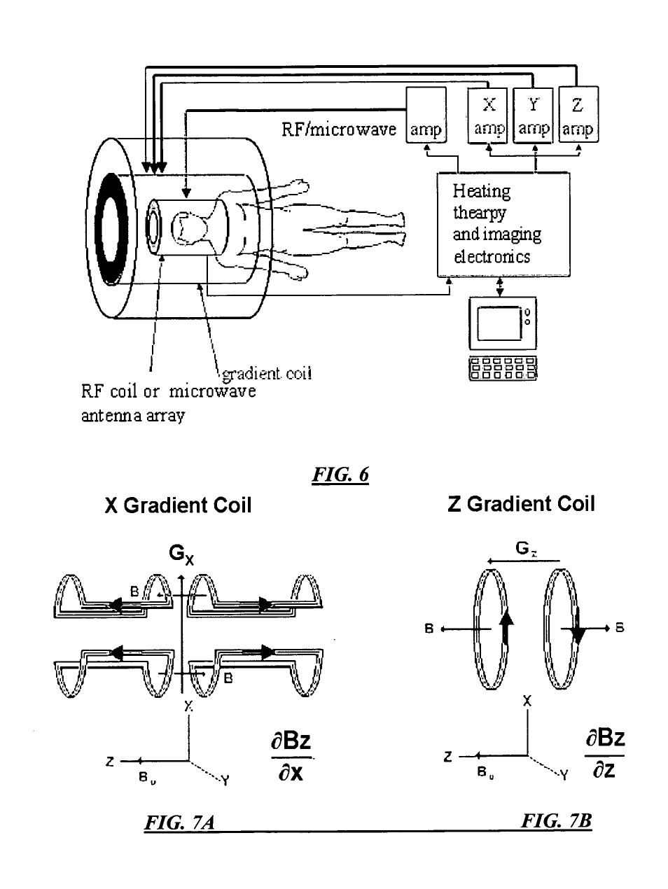

FIG. 7 illustrates typical gradient coils used to generate field gradient along x, y, z directions according to specific embodiments of the invention. The three directions—x, y and z are defined relative to the direction of DC magnetic field, which usually points to z direction. As shown in FIG. 7(b), a Maxwell pair of coils can be used to produce gradient field in z direction (Gz). By adding opposite current to the two circular coils, the net magnetic field contributed by two coils points to the z direction and varies in strength along z. Because the magnetic resonance phenomenon requires the exact match between the frequency of RF excitation pulse generated by the RF coil and the frequency of electron spin resonance, which depends in turn, on the local magnetic field, this pulse will excite the MR signal over a correspondingly narrow range of locations: an imaging slice. To realize 3D MRI, the slice selection by z gradient coil is the first approach of spatial encoding.

The spatial encoding in x, y directions are realized by another type of gradient coils—paired saddle coils, which is shown in FIG. 7(a). The x gradient is formed by current that runs on a cylinder such that the two arcs above are both bringing current around the cylinder in a clockwise direction and those arcs below are bringing current around the cylinder in a counter-clockwise direction. This creates a magnetic field pointing in the z direction that varies in strength along the x direction. For y gradient, this configuration need only be rotated by 90 degrees.

Unlike slice selection in z direction, the x, y gradient (Gₓ, G_{y}) has to be applied in a special pulse sequence to realize the space encoding in x and y directions. There are many different types of pulse sequences that can be applied to the x, y gradient coil to realized different special encoding, which result in the different MRI techniques. Two commonly used spatial encoding methods are called phase encoding and frequency encoding [W. A. Edelstein, J. M. S. Hutchison, G. Johnson, and T. W. Redpath, Phys. Med. Biol. 25, 751 (1980).].

Frequency encoding is realized by adding a gradient field to x-direction (Gx) while collecting the free induction decay (FID) signal by RF coils. Due to the field gradient, the processing frequency of electron spins varies linearly along x-direction. The signal containing all this information can be decomposed into its amplitude and frequency components with a Fourier Transform (FT) algorithm. Knowing the strength of the applied gradient field, allows the system to relate frequency to position and the final result is an image showing the spatial distribution of electron spins in the sample in one-dimension (1D).

The phase encoding is realized by applying gradient at y-direction (G_{y}) for a short period of time. The gradient increases the frequency of precession for a very short time. When the gradient is turned off, the frequency of precession remains constant, but the phase of the spins has changed. The stronger the applied phase encode gradient, the greater the difference in phase between processing spins. By combining with the frequency encoding, the MR signal received will therefore contain phase and frequency information that can be analyzed by the Fourier Transform algorithm. By combining phase and frequency encoding gradients, the spins are spatially labeled within the sample in two dimensions.

Some fast MRI techniques need to apply very fast pulse sequence on the gradient coils to get the 2D information from a single or multi shot of RF excitation pulse. Currently, the most popular fast MRI is the EPI or multi-shot EPI techniques. FIG. 8 illustrates a typical two-shot interleaved epi sequence: a) pulse sequence diagram; b) k-space coverage diagram according to specific embodiments of the invention. In each RF shot, field gradient Gz is applied first to realize the slice selection. A pre-excursion of the blipped gradient is applied on y-direction (phase encoding direction) generate phase shift. A fast and intense oscillation gradient is applied on x-direction. The frequency encoding is realized in every half period of the oscillation. In FIG. 8(a), Gₓ at the first half period is negative; the k-space coverage is a single line along Kₓ from right to left as show in FIG. 8(b). In the second half period, Gₓ is positive; the k-space coverage is a single line from left to right. To realize the phase encoding (equivalent the mapping of K_{y}), a series of blipped gradient pulse is applied to G_{y} when Gₓ is crossing 0. This ensures that the phase encoding is slightly different for each shot, so that every line of k-space is acquired. In the second RF shot, the pre-excursion gradient is increase in length by half of the duration of one blip relative to that in the first RF shot. Consequently the Kₓ and K_{y} trajectory in 1^{st} and 2^{nd} shot form a mesh in K-space. The fully 2D image can be acquired by taking Fourier transform of the sum of the data acquired by first and second shot.

Due to the low magnetic field gradient requirement of eMRI, the conventional gradient coil used for nMRI can be directly used in systems according to specific embodiments of the invention. The coil should have high efficiency, low inductance and low resistance, in order to minimize the current requirements and heat deposition. An important requirement for the gradient coil amplifiers is the maximum current output and the slew rate, which are essential to generate the short and intense current pulse for the spatial encoding. In specific example systems, a commercially available nonlinear amplifier can be used to achieve these specifications.

Thus, as described herein, an eMRI system according to specific embodiments of the invention can increase the spatial resolution, decrease the performance time and lower the cost when compared to nMRI. One eMRI setup can use the same level gradient coils for nMRI to ensure the high performance of the imaging. The slew rate of the nonlinear amplifiers is same too to ensure the short pulse capabilities. However, the maximum current output of the amplifiers can be lower down to 1/10 of the value of nMRI in order to decrease the cost. Even in this case, the eMRI system can still improve the resolution or performance time by a factor of 130.

3) RF Coils, Transmitter and Amplifiers

The RF coils have two functions: (1) Generate the radio frequency pulse that induces the spin resonance of the contrast agent under the magnetic field B₀=2πf/γₑ, where f is the center frequency of the RF pulse; (2) Detect the free induction decay or spin echo of the spin resonance excited by the RF pulse. The RF coils for nMRI can be directly used in the eMRI setup as long as its working frequency is around 1 GHz.

RF coils can be divided into three general categories: (1) transmit and receive coils, (2) receive only coils, and (3) transmit only coils. Transmit and receive coils serve as the transmitter of the B₁ fields and receiver of RF energy from the imaged object. A transmit only coil is used to create the B₁ field and a receive only coil is used in conjunction with it to detect or receive the signal from the spins in the imaged object.

An imaging coil must resonate, or efficiently store energy, at the spin resonance frequency. All imaging coils are composed of an inductor, or inductive elements, and a set of capacitive elements. There are many types of imaging coils. Volume coils surround the imaged object while surface coils are placed adjacent to the imaged object. An internal coil is one designed to record information from regions outside of the coil, such as a catheter coil designed to be inserted into a blood vessel. Some coils can operate as both the transmitter of the B1 field and the receiver of the RF signal. Other coils are designed as only the receiver of the RF signal. In this example system, the eMRI is used for small animal detection; therefore the volume coil is the best choice for this application. Several kinds of volume type RF coils can be used in this setup, such as Alderman-Grant Coil, Bird Cage Coil, Lits Coil, or Saddle Coil.

The transmit/receive switch is added in the RF circuit which allows RF pulse pass through during the transmit time but protects the receiver. This is necessary since the RF pulse is on the order of watts while the MR signal will be on the order of microwatts.

The transition path of the RF circuit contains the low noise RF synthesizer, high power RF amplifier and the logic circuit for RF pulse control. Two kinds of RF pulses are required in eMRI, 90° pulse and the 180° pulse, which rotate the spins along B₁ direction by 90° and 180° respectively. There are two types of spin resonance signal most commonly used in conventional MRI, one is the free induction decay (FID), and the other is the spin echo. FID can be easily detected by apply a 90° pulse to the object. The spin echo signal need to apply a 90° excitation pulse and a 180° echo-forming pulse, resulting in the formation of a Hahn echo during the readout period.

The receiver path of the RF circuit consists of a low-noise RF amplifier and a demodulator to shift the frequency of the signal down to kHz range, a filter to reduce the bandwidth of signal and hence reduce noise.

Similar to the RF coils, the requirement on RF electronics for eMRI is the same as that for the conventional nMRI. All the components are commercially available [www(.usainstruments.com and www.cpcamps.com] and can be directly used.

4) Controller

In this example embodiments, the controller controls all components in the eMRI system in a proper sequence to realize 3D imaging. The basic functions of the controller include:
(1) Controlling the magnet power supply to generate proper DC magnetic field.
(2) Generating pulse sequence to control the x, y, z gradient amplifier for the necessary spatial encoding.
(3) Generating pulse sequence to control RF pulse output.
(4) Generating I/O signal to control the transmitter/receiver switch in the RF circuit.
(5) Converting analog data from RF demodulators to digital signal through high speed AD data acquisition.

Function (1) can be realized by conventional I/O or DA board which is determined by the requirement of the power supply. Functions (3)-(5) can be implemented by specially designed components due to the high performance requirement of eMRI.

One major advantage of eMRI is the reduction in performance time. Short pulse output is required to achieve this goal. In addition, the time interval between pulses has to be short too. As a comparison, we can analyze the performance time of EPI based nMRI as example.

In EPI based nMRI, a typical gradient pulse is about 250 Gauss/m with duration of 0.5 ms/line which result in a spatial resolution of 1.9 mm. For 128 (pixels/line)×128(lines) pixels image, the total spatial encoding time is 64 ms. The protons in human brain has a T₂ of about 100 ms at typical imaging field strengths. Thus, a 64-ms readout is realistic. Further improvement of the performance time can be achieved by increasing the gradient amplitude and shortening the pulse duration without sacrificing the spatial resolution. However, the hardware capabilities on the gradient coil and the amplifiers finally limit the further improvement.

In eMRI, when a gradient pulse is 25 Gauss/m ( 1/10 of nMRI) with duration of 20 μs/line, the spatial resolution can still achieve 0.73 mm which is 2.5 times better than that of nMRI. The performance time for 128×128 pixels image can be decreased to 2.56 ms with single shot EPI method. However the relaxation time T₂ of the contrast agent N@C60 is around 100 μs. Over the course of 2.56 ms for 128 lines data reading duration, the signal will have decayed to nothing. The multi-shot EPI must be used to solve this problem. For example, apply 16 RF pulse pairs (90° and 180° pulse at interval TR of about 80 μs), in each shot, 8 lines of data is read out by applying 20 μs frequency encoding pulse in an oscillating way. The total reading time is 160 μs which is allowed under T₂ limitation. The total 2D imaging time is equals to (160 μs+80 μs)×16=4 ms, which is slightly longer than 2.56 ms in single shot but still much shorter than that of nMRI (64 ms). On the other hand, due to the decrease of gradient field, under same slew rate, shorter gradient pulse can be generated. Under 25 Gauss/m field gradient, a 20 μs pulse is not hard to generate by the amplifiers.

According to the above analysis, eMRI according to specific embodiments of the invention is preferably operated with several requirements on the pulse sequence generators:
a) Capable of generating bipolar pulse with μs level width;
b) Capable generating pulse sequence with time interval controllable in the μs level.
c) Multichannel (at least five channels) output capabilities with synchronization between these channels.

Thus, in specific embodiments, the invention is used with a controller and nanosecond pulse sequence generator as described above.

The single line data contains all the information of frequency encoding. The data acquisition speed needs to be increased accordingly due to the shortening of the gradient pulse. To acquire at least 128 data points within 20 μs as mentioned above, 6.4 MS/s sample rate is required. High performance A/D boards or components available on the market can achieve 400 MS/s sample rate with 100 MHz bandwidth and 12-bit resolution, which is fast enough for this application.

5) Computer and Software

In specific embodiments, the controller is controlled by a PC computer or some other logic processing device or module with control software. Beside the hardware control, the major functions of software are to perform the Fourier transformation and display the 3D images.

### 10. LITERATURE CITED

C. P. Poole, Electron Spin Resonance (2nd Edition), Wiley-Interscience, (1983). T. Almeida Murphy, Th. Pawlik, A. Weidinger, M. Höhne, R. Alcala, and J.-M. Spaeth, Phys. Rev. Lett. 77, 1075 (1996).
C. Knapp, K.-P. Dinse, B. Pietzak, M. Waiblinger, A. Weidinger, Chem. Phys. Lett. 272, 433 (1997).
S. Knorr, A. Grupp, M. Mehring, M. Waiblinger, and A. Weidinger, Electronic Properties of Novel Materials—Molecular Nanostructures, AIP Conference Proceedings 544, 191 (2000).
D. I. Schuster, S. R. Wilson, R. F. Schinazi, Biorg. Med. Chem. Lett. 6, 1253 (1996).
L. Ciobanu, D. A. Seeber, and C. H. Pennington, J. Magn. Reson. 158, 178 (2002).
D. Rugar, R. Budakian, H. J. Mamin, and B. W. Chui, Nature 430, 329 (2004).
J. A. Peters, J. Huskens, D. J. Raber, Progress in Nuclear Magnetic Resonance Spectroscopy 28, 283 (1996).
T. Andersson, K. Nisson, M. Sundahl, G. Westman, and O. Wennerstrom, J. Chem. Soc. Chem. Commun. (8), 604 (1992)
T. Almeida Murphy, Th. Pawlik, A. Weidinger, M. Hohne, R. Alcala, and J.-M. Spaeth, Phys. Rev. Lett. 77, 1075 (1996).
B. Pietzak, M. Waiblinger, T. Almeida Murphy, A. Weidinger, M. Hohne, E. Dietel, A. Hirsch, Chem. Phys. Lett. 279, 259 (1997).
A. Grupp, O. Haufe, M. Jansen, M. Mehring, M. Panthofer, J. Rahmer, A. Reich, M. Rieger, X.-W. Wei, Structure and Electronic Properties of Molecular Nanostructures, AIP, 31 (2002).
M. A. G. Santos, V. Veredas, I. J. Silva Jr., C. R. D. Correia, L. T. Furlan, C. C. Santana, Braz. J. Chem. Eng. 21, No. 1 (2004).
Wilson M W, Hamilton B H, Dong Q, et al, “Gadolinium-enhanced magnetic resonance venography of the portal venous system prior to transjugular intrahepatic portosystemic shunts and liver transplantation”, Investigative Radiology; 33, 644 (1998).
Barkhausen J, M D, Ruehm S G, M D, Goyen M, Buck T, M D, Laub G, Debatin J F, “MR evaluation of ventricular function: true fast imaging with steady-state precession versus fast low-angle shot cine mr imaging: feasibility study”, Radiology 219, 264 (2001).
Carr J C, Simonetti O, Bundy J, PhD, Li D, PhD, Pereles S, Finn J P, “Cine MR Angiography of the Heart with Segmented True Fast Imaging with Steady-State Precession”, Radiology; 219, 828 (2001).
Nghiem H V, Freeny P C, Winter T C 3rd, Mack L A, Yuan C, “Phase-contrast MR angiography of the portal venous system: preoperative findings in liver transplant recipients”, AJR 163, 445.
M. A. G. Santos, V. Veredas, I. J. Silva Jr., C. R. D. Correia, L. T. Furlan, C. C. Santana, Braz. J. Chem. Eng. 21, No. 1 (2004).
www(.)usainstruments(.)com and http://www(.)cpcamps(.)com(.)

### 11. OTHER EMBODIMENTS

In further embodiments, the systems and/or methods as described herein can employ or be used to fabricate a range of paramagnetic endohedral fullerenes and derivatives thereof, and compounds as described herein can in some embodiments be used as spin labels, MRI contrast agents, bio-reporters, and the like. ESR endohedral fullerene spin labels that can be manufactured according to specific embodiments of the invention comprise a fullerene (e.g., C₆₀, C₇₀, C₈₂, C₈₄, C₉₂, C₁₀₆, etc.) containing an atom that when caged within the fullerene is paramagnetic. Some atoms, such as members Group V of the Periodic table (N, P, As, Sb, or Bi) can in theory contribute a paramagnetic spin by chemically bonding to the carbon wall as an ionized “donor” of an electron into the 1 s shell. A similar situation might occur for Group III elements (B, Al, Ga, In, or Tl) acting as ionized “acceptors” to the carbon, creating paramagnetic holes in the 2p shell. Other possibilities, where atomic or ionic radii allow, would include the transition metal series with the magnetic moments of unfilled d shells. These candidates include 3d-series elements Nos. 21 to 29 (Sc, Ti, V, Cr, Mn, Fe, Co, Ni, Cu, and Zn), as well as paramagnetic members of the 4d Nos. 39 to 48 and the 5d series Nos. 71 to 80, all of which can act as a “free” or unbound particle inside the fullerene cage, without constraint by a meaningful chemical bond. Some of the smaller atoms of the Group I alkali metals (Li, Na, K, Rb, or Cs) might also contribute an unpaired electron spin. Members of the lanthanide or “rare earth” series Nos. 57 through 70 with large paramagnetic moments (e,g., _La, Ce, Pr, Nd, Pm, Sm, Eu, Gd, Th, Dy, Ho, Er, Tm, or Yb) form unfilled 4f shells can offer the most attractive possibilities from a sensitivity standpoint, provided that their large radii can be accommodated by the fullerene cages. The only elements that are reasonably excluded are the noble gases of Group VIII, which cannot carry a paramagnetic moment.

Endohedral fullerenes that can be used with systems and methods according to specific embodiments of the invention can be represented by the formula: X@Cₙ where X is the atom caged within the fullerene and Cₙ designates the fullerene (e.g., n can be 60, 70, 82, 84, and so forth).

In certain embodiments the endohedral fullerenes of this invention can be derivatized to increase solubility and/or serum half-life (e.g., with PEG to increase serum half life in vivo). The endohedral fullerenes can also be functionalized with various inorganic or organic targeting moieties (e.g., lectins, antibodies, nucleic acids, chelates, etc.) in order for them to be delivered to and specifically attached to targeted targeted molecules, cells, tissues, viruses, or pathogens, and the like. In certain embodiments, targeting moieties are coupled to an epitope tag or chelate. In certain embodiments, the endohedral fullerenes described herein are coupled to one or more targeting moieties so that they specifically or preferentially bind to certain target(s). In various embodiments the endohedral fullerenes of this invention can be functionalized to accomplish one of more a number of goals. In certain embodiments the fullerenes are derivatized to prevent aggregation. In various embodiments, the endofullerenes are derivatized to increase serum half-life (e.g., to prevent scavenging, chelating, hydrolysis, cellular uptake, immune response, and/or uptake by the RES). Various procedures, methods and techniques known in the art for introducing functional groups onto the fullerene cage of fullerenes or metallofullerenes can be utilized. These embodiments will be more fully understood with reference to the above incorporated U.S. patent applications.

In certain embodiments, the endohedral fullerene spin labels described herein are coupled to one or more targeting moieties so that they specifically or preferentially bind to certain target(s) (e.g., cancer cells). In certain preferred embodiments, the endohedral fullerene(s) are joined to an antibody or to an epitope tag, e.g., through a chelate. The targeting moiety bears a corresponding epitope tag or antibody so that simple contacting of the targeting moiety to the endohedral fullerene(s) results in attachment of the targeting moiety with the endohedral fullerene(s). The combining step can be performed before the targeting moiety is used (targeting strategy) or the target tissue can be bound to the targeting moiety before the endohedral fullerene chelate is delivered. Methods of producing chelates suitable for coupling to various targeting moieties are well known to those of skill in the art. These embodiments will be more fully understood with reference to the above incorporated U.S. patent applications.

### 12. IMAGING REAGENTS FOR ADMINISTRATION TO MAMMALS

The endohedral fullerene spin labels or endohedral fullerene spin labels attached to targeting moieties of this invention (particularly those specific for cancer or other pathologic cells) can be useful for parenteral, topical, oral, or local administration (e.g., injected into a tumor site), aerosol administration, and the like. The imaging compositions can be administered in a variety of unit dosage forms depending upon the method of administration. For example, unit dosage forms suitable for oral administration include powder, tablets, pills, capsules and lozenges. It is recognized that imaging compositions of this invention, when administered orally, can be protected from digestion. This is typically accomplished either by complexing the active component (e.g., the targeting moiety and/or endohedral fullerene spin labels) with a composition to render it resistant to acidic and enzymatic hydrolysis or by packaging the active ingredient(s) in an appropriately resistant carrier such as a liposome. Means of protecting components from digestion are well known in the art.

The imaging compositions of this invention are particularly useful for parenteral administration, such as intravenous administration or administration into a body cavity or lumen of an organ. The compositions for administration will commonly comprise a solution of the endohedral fullerene spin labels and/or endohedral fullerene spin labels attached to targeting moieties dissolved in a pharmaceutically acceptable carrier, preferably an aqueous carrier. A variety of aqueous carriers can be used, e.g., buffered saline and the like. These solutions are sterile and generally free of undesirable matter. These compositions can be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of endohedral fullerene spin labels in these formulations can vary widely, and will be selected primarily based on fluid volumes, viscosities, body weight and the like in accordance with the particular mode of administration selected and the patient's needs.

It will be appreciated by one of skill in the art that there are some regions that are not heavily vascularized or that are protected by cells joined by tight junctions and/or active transport mechanisms which reduce or prevent the entry of macromolecules present in the blood stream

One of skill in the art will appreciate that in these instances, the imaging compositions of this invention can be administered directly to the site. Thus, for example, brain tumors can be visualized by administering the imaging composition directly to the tumor site (e.g., through a surgically implanted catheter).

### 13. KITS

In various embodiments, kits are provided for the practice of this invention. The kits can comprise one or more containers containing endohedral fullerene spin labels as described herein. The endohedral fullerene spin labels can optionally be derivatized, e.g., for attachment to a targeting moiety. In certain embodiments, the endohedral fullerene spin labels are provided already attached to a targeting moiety. In certain embodiments, the endohedral fullerene spin labels and targeting moieties are provided separately and the kit further contains reagents for coupling targeting moieties to the endohedral fullerene spin labels. The kit is preferably designed so that the manipulations necessary to perform the desired reaction should be as simple as possible to enable the user to prepare from the kit the desired composition by using the facilities that are at his disposal. Therefore the invention also relates to a kit for preparing a composition according to this invention. In certain embodiments, the kit can optionally, additionally comprise a reducing agent and/or, if desired, a chelator, and/or instructions for use of the composition and/or a prescription for reacting the ingredients of the kit to form the desired product(s). If desired, the ingredients of the kit may be combined, provided they are compatible.

In certain embodiments, the kit components (e.g., endohedral fullerene spin labels) are preferably sterile and can, optionally be provided in a pharmacologically acceptable excipient. When the constituent(s) are provided in a dry state, the user should preferably use a sterile physiological saline solution as a solvent. If desired, the constituent(s) can be stabilized in the conventional manner with suitable stabilizers, for example, ascorbic acid, gentisic acid or salts of these acids, or they may comprise other auxiliary agents, for example, fillers, such as glucose, lactose, mannitol, and the like.

In certain embodiments, the kits additionally comprise instructional materials teaching the use of the compositions described herein (e.g., endohedral fullerene spin labels, derivatized endohedral fullerene spin labels, etc.) in electron spin resonance applications for selectively imaging cells, tissue, organs, and the like.

While the instructional materials, when present, typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### 14. EMBODIMENT IN A PROGRAMMED INFORMATION APPLIANCE

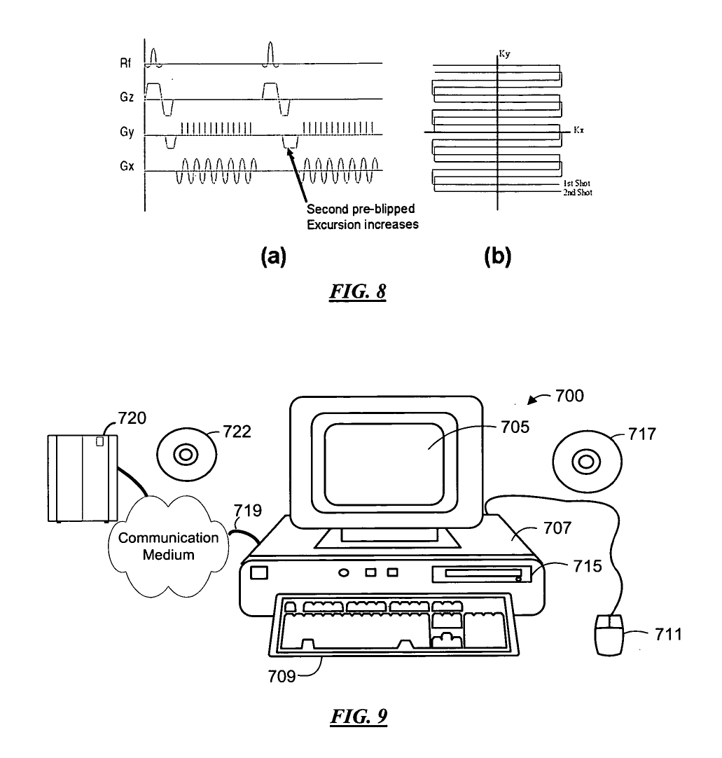

FIG. 9 is a block diagram showing a representative example logic device in which various aspects of the present invention may be embodied. As will be understood to practitioners in the art from the teachings provided herein, the invention can be implemented in hardware and/or software. In some embodiments of the invention, different aspects of the invention can be implemented in either client-side logic or server-side logic. As will be understood in the art, the invention or components thereof may be embodied in a fixed media program component containing logic instructions and/or data that when loaded into an appropriately configured computing device cause that device to perform according to the invention. As will be understood in the art, a fixed media containing logic instructions may be delivered to a user on a fixed media for physically loading into a user's computer or a fixed media containing logic instructions may reside on a remote server that a user accesses through a communication medium in order to download a program component.

FIG. 9 shows an information appliance (or digital device) 700 that may be understood as a logical apparatus that can read instructions from media 717 and/or network port 719, which can optionally be connected to server 720 having fixed media 722. Apparatus 700 can thereafter use those instructions to direct server or client logic, as understood in the art, to embody aspects of the invention. One type of logical apparatus that may embody the invention is a computer system as illustrated in 700, containing CPU 707, optional input devices 709 and 711, disk drives 715 and optional monitor 705. Fixed media 717, or fixed media 722 over port 719, may be used to program such a system and may represent a disk-type optical or magnetic media, magnetic tape, solid state dynamic or static memory, etc. In specific embodiments, the invention may be embodied in whole or in part as software recorded on this fixed media. Communication port 719 may also be used to initially receive instructions that are used to program such a system and may represent any type of communication connection. It will also be understood that functional components of such a computer system can be incorporated into various integrated laboratory systems, such as a turn-key eMRI system according to specific embodiments of the invention.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

All publications, patents, and patent applications cited herein or filed with this application, including any references filed as part of an Information Disclosure Statement of this or incorporated applications at the time of filing this application, are incorporated by reference in their entirety.

What is claimed:

1. A method for imaging a solid body or object comprising:
using a contrast agent with electron spin resonance (ESR) relaxation times (e.g., T₁ and T₂) that are comparable to nuclear resonance relaxation times of NMR materials (e.g., protons in water);
using a magnetic resonance imaging (MRI) technique based on nuclear magnetic resonance to realize electron spin resonance imaging (eMRI).

2. The method of claim 1 further comprising:
wherein said contrast agent is an endohedral fullerene.

3. The method of claim 1 further wherein said contrast agent is a nitrogen endohedral fullerene (N@C₆₀).

4. The method of claim 1 further wherein said contrast agent is an endohedral fullerene X@Cₙ, wherein:
X is a paramagnetic material; and
n is selected from the group consisting of 60, 70, 82, 84, 92, and 106.

5. The method of claim 1 further comprising:
using a higher resonance frequency to drive said ESR, thereby achieving higher sensitivity when compared to NMR.

6. The method of claim 1 further comprising:
using a lower powered magnetic field, thereby allowing for lower cost imaging.

7. The method of claim 1 further comprising:
achieving higher spatial resolution using said lower magnetic field when a field gradient is kept constant.

8. The method of claim 1 further comprising:
achieving shorter performance time due to using said lower magnetic field when a field gradient is kept constant.

9. The method of claim 1 wherein performance time and spatial resolution of said eMRI is governed by:
$$Δ X = \frac{1}{γ \cdot t \cdot G} = \frac{{B}_{0}}{ω \cdot t \cdot G} \qquad ( 3 )$$
where
where γ is the gyromagnetic ratio,
ω is the MR frequency, and B₀ is the required DC magnetic field.
wherein the gyromagnetic ratio of electron spin is approximately 650 times higher than that of the proton in nMRI.

10. The method of claim 1 wherein a larger gyromagnetic ratio of electron spin relative to a gyromagnetic ratio of proton spin in nMRI allows for:
a one order of magnitude increase in imaging frequency; and
increased sensitivity of eMRI by two orders of magnitude.

11. The method of claim 1 further comprising:
wherein when using a higher frequency ˜1 GHz, it is possible to lower said magnetic field by about two orders of magnitude (e.g., to 350 Gauss compared to that of nMRI which is most commonly ˜3.9T);

12. The method of claim 1 further comprising use of a lowered B₀ field allowing:
decrease in performance time of eMRI by about two orders of magnitude when the spatial resolution and the gradient field amplitude are kept the same as nMRI;

13. The method of claim 1 further comprising use of a lowered B₀ field allowing:
increased spatial resolution of eMRI can by two orders of magnitude when the performance time and the gradient field amplitude are kept the same as nMRI;

14. The method of claim 1 further comprising use of a lowered B₀ field allowing:
decreased gradient field amplitude required for eMRI by two orders of magnitude when the spatial resolution and the performance time are kept the same as nMRI, which significantly lowers the cost;

15. The method of claim 1 further comprising use of a lowered B₀ field allowing:
decrease in performance time of eMRI by about one order of magnitude and decrease the gradient field amplitude required for eMRI by one order of magnitude to lower the cost of the instrument, while keeping spatial resolution the same as nMRI.

16. A method for ESR imaging comprising:
using a spin resonance frequency of up to 1 GHz with a several hundred Gauss magnetic field;
wherein due to the increase of the resonance frequency and therefore the increase of spin population difference, the sensitivity of ESR contrast agents increases by at least one order of magnitude, which gives ˜10¹¹ to 10¹³ electron spins sensitivity;
wherein whole body MRI resolution is around 1 mm in size;
further wherein to reach the same resolution, the concentration of ESR contrast agents is determined by:
$$C = \frac{{10}^{11} \sim {10}^{13}}{{( 1 mm )}^{3}} = 1.6 \times {10}^{- 7} \sim 1.6 \times {10}^{- 5} mol / L \qquad ( 4 )$$
which required concentration is one to three order of magnitude lower than regular MRI contrast agent concentration (0.1 to 0.5 mmol/L).

17. A method of fabricating endohedral fullerenes with a paramagnetic material with high concentration levels comprising:
inductively inducing an appropriate (X) ion plasma inside a sealed compartment filled with a high concentration of Cₙ molecule vapor.
wherein:
X is said paramagnetic material; and
n is selected from the group consisting of 60, 70, 82, 84, 92, and 106.

18. A method of fabricating endohedral fullerenes with high concentration levels comprising:
sealing Cₙ powder and X gas within a compartment, which is surrounded with an RF coil;
cooling one end of the compartment by liquid X to condense X gas inside the compartment while keeping pressure inside the compartment lower than atmosphere;
heating the compartment;
such that solid Cₙ will vaporize filling the compartment and inductively induced X ions will collide with Cₙ molecules continuously in the process;
wherein:
X is said paramagnetic material; and
n is selected from the group consisting of 60, 70, 82, 84, 92, and 106,
said endohedral fullerenes represented by a formula X@Cₙ.

19. The method of claim 18 further comprising:
wherein said heating is to about 450° C.

20. The method of claim 18 wherein X is nitrogen.

21. The method of claim 18 wherein said compartment is a quartz tube.

22. The method of claim 18 wherein said compartment is a glass tube.

23. The method of claim 18 wherein X is not nitrogen.

24. The method of claim 18 wherein said fullerene cages a single atom.

25. The method of claim 18 wherein said fullerene cages two atoms.

26. The method of claim 18 wherein X comprises a material that has an electron spin resonance (ESR) Q greater than 10, when caged within said fullerene.

27. The method of claim 18 wherein X comprises a material that has an electron spin resonance (ESR) Q ranging from about 100 to about 1000 when caged within said fullerene.

28. The method of claim 18 wherein X is selected from the group consisting of N, P, As, and a lanthanide.

29. The method of claim 18 wherein the longer the system is operated, the higher concentration of X@Cₙ are obtained;
wherein inductively induced ion plasma (instead of high electric field induced plasma as in previous study) reduces the chance of fracturing Cₙ in the process.

30. The method of claim 18 further comprising:
allowing said compartment to cool;
extracting endohedral fullerene powder (mixed with empty Cₙ) from said compartment by an appropriate chemical solution (e.g., toluene or hexane).

31. A method of fabrication of endohedral fullerenes with high concentration levels comprising:
inductively inducing Nitrogen ion plasma inside a sealed glass tube filled with high concentration of C₆₀ molecule vapor;
sealing C₆₀ powder and N₂ gas within a quartz (or glass) tube, which is surrounded with a RF coil;
cooling one end of the tube by liquid N₂ to condense N₂ gas inside the tube while keeping the pressure inside the tube lower than atmosphere;
heating to about 450° C.;
such that solid C₆₀ will vaporize ° C. filling the entire tube, and inductively induced nitrogen ions will collide with C₆₀ molecules continuously in the process;
wherein the longer the system is operated, the higher concentration of N@C₆₀ are obtained;
wherein inductively induced ion plasma (instead of high electric field induced plasma as in previous study) reduces the chance of fracturing C₆₀ in the process;
allowing said tube to cool;
extracting nitrogen endohedral fullerene powder (mixed with empty C₆₀) from said quartz tube by an appropriate chemical solution (e.g., toluene or hexane).

32. A method of separating X@Cₙ from pure Cₙ comprising:
using simulated moving bed (SMB) chromatography to purify endohedral fullerenes from empty fullerenes;
wherein SMB chromatography is a continuous solid-liquid separation process that purifies two components of a feed stock;
with efficient use of separations packing and eluant and high productivity.
wherein:
X is a paramagnetic material; and
n is selected from the group consisting of 60, 70, 82, 84, 92, and 106.
said endohedral fullerenes represented by a formula X@Cₙ.

33. The method of claim 32 further comprising:
after purification, reusing empty fullerenes to produce endohedral fullerenes.

34. The method of claim 31 further comprising:
separating N@C₆₀ from pure C₆₀ using simulated moving bed (SMB) chromatography to purify endohedral fullerenes from empty fullerenes;
reusing empty fullerenes in a further N@C₆₀ fabrication.

35. A nano-second pulse sequence generator designed for electron spin echo observation:
wherein both pulse width and time interval between pulses can be adjusted from 1 ns with 10 ps resolution.

36. An eMRI system using nMRI encoding methods and providing higher image acquisition rate and higher sensitivity (or lower contrast agent dosage) with the same spatial resolution as nMRI comprising:
a magnet for producing a stable magnetic field;
gradient coils for creating a variable field;
radio frequency (RF) coils used to transmit energy and to encode spatial position;
electronics that drive the magnet and coils; and
computer controlled scanning operation and data processing.

37. The system of claim 36 further wherein:
because the eMRI needs magnetic field of several hundred gauss, magnetic shielding is required to avoid the disturbance from surrounding environment.

38. The system of claim 36 further wherein:
a DC magnetic field generated by the magnet system determines the frequency of magnetic resonance, such that when the frequency is about 1 GHz, the required magnetic field is about 350 Gauss.

39. The system of claim 36 further wherein:
said magnet can be one or more of:
a permanent magnet;
an electromagnet;
wherein an electromagnet can be used to provide an adjustable magnetic field;
wherein said electromagnet is one of: (1) an iron core electromagnet, or (2) an air core solenoid electromagnet.
for high quality ESR imaging, the DC magnetic field has to be very uniform in the entire detection region to ensure the high signal noise ratio and avoid the image distortion.

40. The system of claim 36 further wherein:
a custom-designed air core solenoid electromagnet is used to generate more uniform magnetic field inside the solenoid;
wherein to generate 350 Gauss, the coil current density is 30 kA/m;
wherein said current density is achieved by adding a water cooling feature.

41. The system of claim 36 further wherein:
gradient coils are used to produce a linear variation in field along 3 directions respectively;
wherein spatial encoding in x, y directions are realized by another type of gradient coils paired saddle coils.

42. The system of claim 36 further wherein:
due to the low magnetic field gradient requirement of eMRI, a conventional gradient coil used for nMRI can be directly used wherin said coil has high efficiency, low inductance and low resistance, in order to minimize the current requirements and heat deposition.

43. The system of claim 36 further wherein:
gradient coil amplifiers have a sufficient maximum current output and slew rate to generate the short and intense current pulse for the spatial encoding;
wherein commercially available nonlinear amplifier can be used achieve required specifications.
wherein the maximum current output of the amplifiers can be lower down to 1/10 of the value of nMRI in order to decrease the cost, while eMRI still provides improved resolution or performance time by a factor of 130.

44. The system of claim 36 further wherein:
RF coils for nMRI can be used as long as a working frequency is around 1 GHz;
an RF circuit transition path comprising a low noise RF synthesizer, high power RF amplifier and logic circuit for RF pulse control able to provide two kinds of RF pulses: 90° pulse and the 180° pulse, which rotate the spins along B1 direction by 90° and 180° respectively.
a RF circuit receiver path comprising a low-noise RF amplifier and a demodulator to shift the frequency of the signal down to kHz range, a filter to reduce the bandwidth of signal and hence reduce noise;
thereby allowing RF coils and RF electronics as used in conventional nMRI for eMRI

45. The system of claim 36 further comprising:
a controller to control components in the eMRI system in a proper sequence to realize 3D imaging, said controller performing:
(1) Controlling the magnet power supply to generate proper DC magnetic field.
(2) Generating pulse sequence to control the x, y, z gradient amplifier for the necessary spatial encoding.
(3) Generating pulse sequence to control RF pulse output.
(4) Generating I/O signal to control the transmitter/receiver switch in the RF circuit.
(5) Converting analog data from RF demodulators to digital signal through high speed AD data acquisition.

46. The system of claim 45 further comprising:
pulse sequence generators capable of:
generating bipolar pulse with Us level width;
generating pulse sequence with time interval controllable in the us level;
Multichannel (e.g., at least five channels) output capabilities with synchronization between these channels.

47. The system of claim 46 further wherein:
said controller uses a proprietary nanosecond pulse sequence generator.

48. A kit for selectively imaging a cell or tissue, said kit comprising:
a container containing a composition comprising a paramagnetic material caged within a fullerene.

49. The kit of claim 48, wherein said paramagnetic material caged within a fullerene has the formula
X@Cₙ
wherein:
X is said paramagnetic material; and
n is selected from the group consisting of 60, 70, 82, 84, 92, and 106.
